(19) United States
(12) Patent Application Publication
(10) Pub. No.: US 2018/0325454 A1
(43) Pub. Date: Nov. 15, 2018

(54) CONFORMABLE BIOMECHANICAL FORCE SENSOR AND METHOD OF FABRICATION

(71) Applicant: University of Utah Research Foundation, Salt Lake City, UT (US)

(72) Inventors: Tomasz J. Petelenz, Salt Lake City, UT (US); Robert W. Hitchcock, Salt Lake City, UT (US); Erika Vandersteen, Farmington, UT (US)

(21) Appl. No.: 15/776,910

(22) PCT Filed: Nov. 17, 2016
(86) PCT No.: PCT/US2016/062533

§ 371 Date: May 17, 2018

Related U.S. Application Data
(60) Provisional application No. 62/257,118, filed on Nov. 18, 2015.

(51) Int. Cl. A61B 5/00; A61B 5/103; A61F 2/76

(52) U.S. Cl. CPC A61B 5/6812; A61B 5/1038; A61B 5/447; A61F 2002/501; A61B 2562/0247; A61B 2562/168; A61F 2/76

(57) ABSTRACT

Sensing devices for measuring a force applied by a patient's body include a first surface and a second surface that form a chamber housing electrical components and an incompressible fluid. The second surface is convex away from the first surface, which is planar. The electrical components include a pressure sensor and a signal conditioner and the incompressible fluid fills the chamber and contacts the electrical components.

### CROSS-REFERENCE TO RELATED APPLICATIONS

This application claims priority to and benefit of U.S. Provisional Patent Application No. 62/257,118, filed Nov. 18, 2015, the entire content of which is incorporated by reference herein.

### BACKGROUND OF THE DISCLOSURE

Bone fractures, or broken bones, may be caused by direct or indirect forces applied to a bone, or as a result of certain medical conditions, such as osteoporosis. Falls, sports injuries, motor vehicle accidents, and other impacts are common causes of bone fractures. Fractures can be very painful, can require significant time to heal, can involve complicated rehabilitation regimen, and can result in significant direct and indirect costs.

For example, the tibia, which is the most commonly broken long bone in the body, typically requires between about ten weeks and about ten months to heal completely, without complications. By some estimates, the number and severity of complications associated with tibial fractures results in an annual direct cost for the United States of about $1.2 billion USD. When indirect costs such as lost wages are factored in, the long rehabilitation period for tibial fracture patients results in an estimated annual indirect cost of about $95 billion USD.

The mechanical environment experienced by the recovering bone is a major factor in fracture healing rate. In an attempt to produce an optimal mechanical environment that promotes bone healing while reducing risks of complications, clinician routinely prescribe PWB during fracture rehabilitation. For example, PWB is commonly prescribed during rehabilitation of hip and lower extremity injuries, such as fractures to hips, femurs, tibia, ankles, calcanei, metatarsals, and the like.

The PWB prescription for a patient varies based on the type and extent of the injury and on the discretion of the clinician. Unfortunately, little data has been collected to support a conclusion that PWB prescriptions are effective at either promoting fracture healing or reducing the risk of complications. Additionally, the patients' tendencies or abilities to comply with the PWB prescription for the entire duration between follow-up visits are unknown. Therefore, clinicians and researchers would greatly benefit from a load monitoring device that can continually track the PWB behavior of a patient between follow-up visits. As follow-up visits may be scheduled one day, a week, or even two-weeks apart, clinicians and researches would greatly benefit from a robust monitoring device capable of tracking the PWB behavior even over extended periods of time.

Other therapeutic processes require the immobilization of the injured bone, extremity, or other body part relative to the patient's body. For example, rehabilitation of a soft-tissue injury to the shoulder, elbow, knee, or other body part may include immobilization of the limb relative to the patient's body using a cast or other temporary immobilization device. The immobilization device may be compressed around the limb to restrict or prevent the movement of the limb. The amount of compression may be prescribed within a particular range to restrict movement without further damaging the limb, either through excessive compression to the bone or to the soft-tissue. The compression is conventionally approximated to be fixed from upon applying the immobilization device to removal, however the precise amount of compression when applying the immobilization device and the amount of compression experienced during wear is unmonitored.

Similarly, prosthetic devices may be worn by a patient by compressing the prosthetic against a portion of the patient's body. The prosthetic is conventionally worn as tightly as possible without damaging the soft tissue, such as constricting blood flow or damaging blood vessels, which is determined by patient feedback.

### BRIEF SUMMARY OF THE DISCLOSURE

This summary is provided to introduce a selection of concepts that are further described below in the detailed description. This summary is not intended to identify specific features of the claimed subject matter, nor is it intended to be used as an aid in limiting the scope of the claimed subject matter.

In an embodiment, a sensing device includes a chamber defined by one or more membranes with a pressure sensor and a signal conditioner located in the chamber. The pressure sensor is in electrical communication with the signal conditioner. The sensing device includes an incompressible fluid in the chamber contacting at least one side of the pressure sensor and the one or more membranes.

In another embodiment, a sensing device includes a rigid first surface and a flexible second surface that is at least partially convex relative to the rigid first surface. The rigid first surface and flexible second surface define a chamber therebetween and bounded by the rigid first surface and the flexible second surface. A pressure sensor is located in the chamber and the chamber contains an incompressible fluid that contacts at least one side of the pressure sensor, the rigid first surface, and the flexible second surface.

In a further embodiment, a therapeutic device includes a support member and a plurality of sensing devices. The support member includes a plurality of wells therein and the pluralities of sensing devices are located in the wells. The sensing devices are in electrical communication with one another. At least one of the sensing devices includes a rigid first surface and a flexible second surface that is at least partially convex relative to the rigid first surface. The rigid first surface and flexible second surface define a chamber therebetween and bounded by the rigid first surface and the flexible second surface. A pressure sensor is located in the chamber and the chamber contains an incompressible fluid that contacts at least one side of the pressure sensor, the rigid first surface, and the flexible second surface.

Additional features of embodiments of the disclosure will be set forth in the description which follows. The features of such embodiments may be realized by means of the instruments and combinations particularly pointed out in the appended claims. These and other features will become more fully apparent from the following description and appended claims, or may be learned by the practice of such exemplary embodiments as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is a perspective view of a side cross-section of an embodiment of a sensing device, according to the present disclosure;

FIG. 2 is a side view of a side cross-section of another embodiment of a sensing device, according to the present disclosure;

FIG. 3 is a perspective view of an embodiment of a sensing device in a well, according to the present disclosure;

FIG. 4 is a perspective view of an embodiment of a therapeutic device including a plurality of sensing devices, according to the present disclosure;

FIG. 5 is a perspective view of an embodiment of a therapeutic device including a plurality of sensing devices, according to the present disclosure;

FIG. 6 is a top view of the embodiment of a therapeutic device including a plurality of sensing devices of FIG. 5, according to the present disclosure;

FIG. 7 is a side view of another embodiment of a therapeutic device including a plurality of sensing devices, according to the present disclosure;

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to constructing and installing sensing devices for use in a therapeutic device, such as a PWB device, an immobilization device, prosthetic device, or combinations thereof. More particularly, one or more embodiments of the present disclosure may relate to a sensing device embedded in and protruding from a flexible or padded surface of a therapeutic device.

One or more embodiments of a sensing device according to the present disclosure may include a pressure sensor housed in a chamber formed by a rigid first surface and a flexible second surface. For example, the rigid first surface and flexible second surface may substantially oppose one another and define at least two sides of the chamber. In some embodiments, the rigid first surface and flexible second surface may be connected to one another by a sidewall extending between the rigid first surface and flexible second surface. In at least one example, the sidewall may be a cylindrical sidewall extending from the rigid first surface to the flexible second surface. In other embodiments, the rigid first surface and flexible second surface may be connected to one another directly. In at least one example, the rigid first surface may be substantially planar and the flexible second surface may be convex away from the rigid first surface and connected at an edge of the flexible second surface to the rigid first surface.

A pressure sensor may be located in the chamber and be in contact with an incompressible gel that may transmit pressure applied to the rigid first surface and/or flexible second surface to the pressure sensor. In some embodiments, the pressure sensor may be in electrical communication with a signal conditioner. The signal conditioner may be housed inside the chamber with the pressure sensor. In at least one embodiment the signal conditioner may be encapsulated in the incompressible gel. In at least one other embodiment, the rigid first surface may be a hybrid circuit or “system-on-chip” containing at least the pressure sensor and the signal conditioner.

In some embodiments, an embodiment of a sensing device according to the present disclosure may be fixed in a therapeutic device, allowing for more reliable measure of pressures applied to the therapeutic device with increased signal-to-noise ratio compared to conventional therapeutic devices.

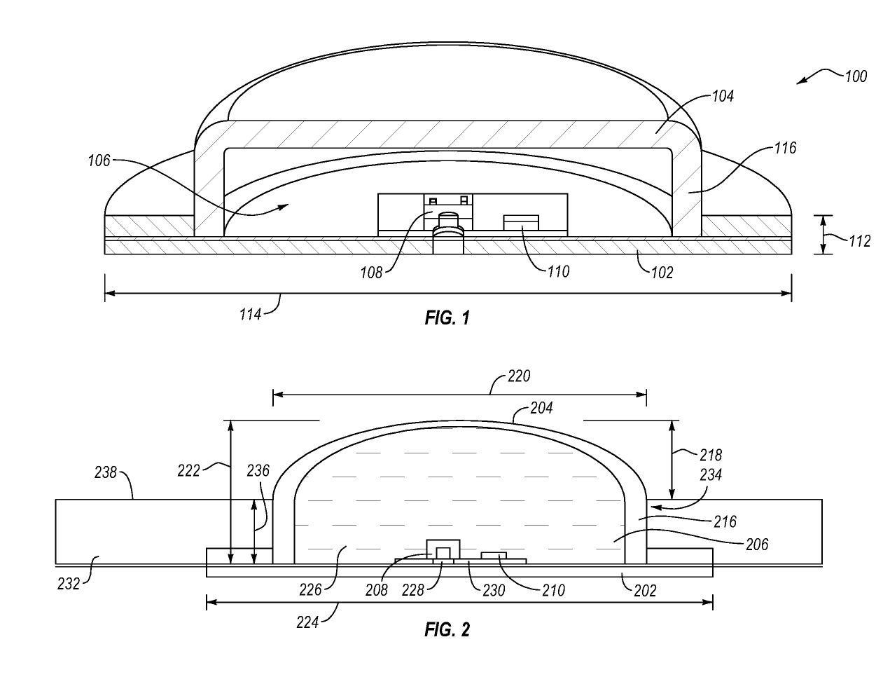

FIG. 1 illustrates an embodiment of a sensing device 100 in accordance with the present disclosure. The sensing device 100 may be bounded by one or more membranes, such as a first surface 102 and a second surface 104. The first surface 102 and second surface 104 may at least partially define a chamber 106 therebetween. A pressure sensor 108 may be located in the chamber 106. In some embodiments, the pressure sensor 108 may be positioned in contact with the first surface 102. In other embodiments, the pressure sensor 108 may be positioned in contact with the second surface 104. In yet other embodiments, the pressure sensor 108 may be positioned within the chamber 106 and not contacting the first surface 102 or the second surface 104 (e.g., suspended between the first surface 102 and second surface 104).

In some embodiments, the pressure sensor 108 may be a piezoresistive sensor. In other embodiments, the pressure sensor 108 may be a force-sensitive resistor sensor, magnetic resistance sensor, strain gauge, spring based sensor, fiber optic based sensor, polarized light sensor, mechanical actuator based sensor, displacement based sensor, any other sensor type capable of measuring a force applied thereto, or combinations thereof. In at least one example, the pressure sensor 108 may be a Wheatstone bridge sensor.

The pressure sensor 108 may be in electrical communication with a signal conditioner 110. The signal conditioner 110 may receive a pressure signal from the pressure sensor 108 and alter the pressure signal. In some embodiments, the signal conditioner 110 may include an amplifier that may amplify at least a portion of the pressure signal. In other embodiments, the signal conditioner 110 may include a filter (i.e., a high-pass filter, low-pass filter, mid-range filter, or combinations thereof) that may filter at least a portion of the pressure signal. In yet other embodiments, the signal conditioner 110 may include a converter that may convert the pressure signal from analog to digital or from digital to analog. In further embodiments, the signal conditioner 110 may include an isolator that may isolate at least a portion of the pressure signal to pass the isolate portion of the pressure signal to another device without a physical connection (e.g., through a magnetic field.) In yet further embodiments, the signal conditioner 110 may alter the pressure signal in other ways.

The signal conditioner 110 being within the sensing device 100 may allow a short transmission distance from the pressure sensor 108 to the signal conditioner 110 in comparison to a signal conditioner 110 housed outside of the chamber 106. For example, the short transmission distance may allow the pressure sensor 108 to transmit the pressure signal to the signal conditioner 110 with less degradation of the pressure signal during transmission. In other examples, the short transmission distance may allow for lower energy consumption and collection of more data in a comparable period of time. In at least one example, a sensing device 100 according to the present disclosure may be capable of sampling the pressure signal from the pressure sensor 108 and conditioning the signal at the signal conditioner 110 at a rate of approximately 60 Hertz for 2 or more weeks at a time. In some embodiments, the sensing device 100 may transmit the data from the sensing device to a data storage device or other computing device, as described in greater detail herein.

In some embodiments, at least a portion of the first surface 102 of the sensing device 100 may be rigid. In other embodiments, the first surface 102 of the sensing device 100 may be a rigid surface. In yet other embodiments, the first surface 102 of the sensing device 100 may be a flexible surface. In at least one embodiment, the first surface 102 may be a rigid surface that is substantially planar. In some embodiments, the first surface 102 may be a rigid surface that has a curvature with a height 112 of no more than 10% of a width 114 of the first surface 102. In other embodiments, the first surface 102 may be a rigid surface that has a curvature with a height 112 of no more than 8% of a width 114 of the first surface 102. In yet other embodiments, the first surface 102 may be a rigid surface that has a curvature with a height 112 of no more than 5% of a width 114 of the first surface. The first surface 102 may have various shapes. In some embodiments, the first surface 102 may be elliptical. In other embodiments, the first surface 102 may be rectangular, triangular, octagonal, other polygonal, circular, irregular, or combinations thereof.

In the rigid embodiments, the first surface 102 may be made of or include metals, metal alloys, rigid plastics, ceramics, organic materials, or combinations thereof.

The second surface 104 may be a flexible surface. The flexible second surface may allow the movement of at least a portion of the second surface 104 upon receiving an external force. In some examples, the flexible second surface 104 may be connected directly to the first surface 102 and may move relative to the first surface 102. In other examples, the flexible second surface 104 may be connected to the first surface 102 by a sidewall 116. The sidewall 116 may, in some embodiments, be a rigid sidewall 118, allowing the flexible second surface 104 to deflect from an initial position relative to the first surface 102. For example, the flexible second surface 104 may act like a diaphragm and the rigid sidewall 116 may be substantially stationary relative to the first surface 102. In other embodiments, the sidewall 116 may be a flexible sidewall 116 that allows displacement of the second surface 104 relative to the first surface 102. The second surface 104 may have various shapes. In some embodiments, the second surface 104 may be elliptical. In other embodiments, the second surface 104 may be rectangular, triangular, octagonal, other polygonal, circular, irregular, or combinations thereof. In some embodiments, the first surface 102 and second surface 104 may be the same shape. In other embodiments, the first surface 102 and second surface 104 may be different shapes, such as an elliptical second surface 104 connected to a rectangular first surface 102, such as depicted in FIG. 1. In at least one embodiment, the first surface 102 and second surface 104 are the same shape and size such that a peripheral edge of the first surface 102 aligns with a peripheral edge of the second surface 104.

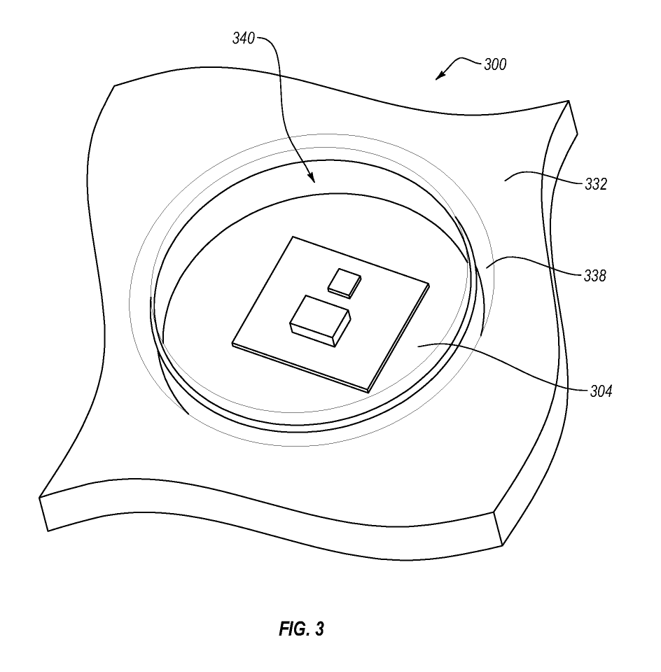

In at least one embodiment, the second surface 104 may be a flexible surface with at least a portion that is substantially planar, such as shown in FIG. 1. As shown in FIG. 2, in some embodiments, a sensing device 200 may have a second surface 204 that is a flexible surface that has a convex curvature relative to a first surface 202 with a height 218 of no more than 100% of a width 220 of the second surface 204. In other embodiments, the second surface 204 may be a flexible surface that has a convex curvature relative to the first surface 202 with a height 218 of no more than 50% of a width 220 of the second surface 204. In yet other embodiments, the second surface 204 may be a flexible surface that has a convex curvature relative to the first surface 202 with a height 218 of no more than 20% of a width 220 of the second surface 204. In further embodiments, the second surface 204 may be a flexible surface that has a convex curvature relative to the first surface 202 with a height 218 of no more than 10% of a width 220 of the second surface 204. In yet further embodiments, the second surface 204 may be a flexible surface that has a convex curvature relative to the first surface 202 with a height 218 of no more than 5% of a width 220 of the second surface 204.

In other embodiments, the sensing device 200 may have a height 222 that is a percentage of a width 224 of the sensing device 200 in a range having upper and lower values including any of 10%, 20%, 30%, 40%, 50%, 60%, 70%, 80%, 90%, 100%, or any value therebetween. For example, the height 222 of the sensing device 200 may be in a range of 10% to 100% of the width 224 of the sensing device 200. In other examples, the height 222 of the sensing device 200 may be in a range of 20% to 80% of the width 224 of the sensing device 200. In yet other examples, the height 222 of the sensing device 200 may be in a range of 30% to 60% of the width 224 of the sensing device 200. In at least one example, the sensing device 200 may be about 15 millimeters in width 224 and about 5 millimeters in height 222.

In the flexible embodiments, the second surface 204 may be made of or include elastomers, resilient metals and/or metal alloys, flexible plastics, organic materials, or combinations thereof.

In other embodiments, the second surface 204 may be a rigid surface and may move relative to the first surface 202 upon receiving an external force. For example, a rigid second surface 204 may be connected to a rigid first surface 202 by a flexible sidewall 216. The flexible sidewall 216 may deform upon receiving a compressive force from the second surface 204. Deformation of the flexible sidewall 216 may allow displacement of the rigid second surface 204 relative to the rigid first surface 202. In another example, the rigid second surface 204 may be slidably connected to a rigid sidewall 216, and the rigid sidewall 216 may be affixed to the rigid first surface 202. The rigid second surface 202 may slide relative to the rigid sidewall 216 (e.g., a piston and cylinder), thereby allowing displacement of the second surface 204 relative to the first surface 202.

A chamber 206 of the sensing device 200 may be various shapes and/or sizes. In some embodiments, the chamber may be defined by the sidewall 216 connecting the first surface 202 and the second surface 204. In other embodiments, the chamber may be defined by the first surface 202 and second surface 204 without a sidewall 216, where the first surface 202 and second surface 204 are connected directly to one another. For example, the first surface 202 and second surface 204 may be connected at a seal. The seal may be formed by welding (such as thermal, mechanical, or acoustic welding), adhesives, mechanical fasteners, a compression fit, a friction fit, a snap fit, other connection mechanisms, or combinations thereof.

The chamber 206 of the sensing device 200 may contain one or more electrical components. In some embodiments, the chamber 206 may contain the pressure sensor 208. In other embodiments, the chamber 206 may contain the signal conditioner 210. In yet other embodiments, the chamber 206 may contain a power supply. The electrical components in the chamber 206 may be in electrical communication with one another via wires. In at least one embodiment, a plurality of electrical components may be attached to the first surface 202 and connected through a wire attached to and/or embedded in the first surface 202.

The chamber 206 may contain a fluid 226 therein, which may transmit a fluid pressure throughout the chamber. In some embodiments, the fluid 226 may be an incompressible fluid, such as a liquid or gel. In other embodiments, the fluid 226 may be a gel that is at least partially cured. For example, the fluid 226 may have a bulk modulus equal to or less than 4.6×10¹⁰ square meters per Newton. In other embodiments, the fluid 226 may be have a stable liquid form in an operating range between −50° and 100° Celsius. The fluid 226 may be in contact with at least one side of the pressure sensor 208 and may apply the fluid pressure thereto. In some embodiments, the pressure sensor 208 may be positioned on the first surface 202 and in communication with an aperture 228 through the first surface 202 to allow relative measurement of the fluid pressure inside the chamber 206 and the atmospheric pressure outside the chamber 206. In other embodiments, the fluid 226 may completely surround and/or encapsulate the pressure sensor 208.

The fluid 226 may contact at least one surface of the signal conditioner 210 inside the chamber 206. For example, the signal conditioner 210 may be connected to the first surface 202 on a first side of the signal conditioner 210 and the fluid 226 may contact an opposing side of the signal conditioner 210. In another example, the signal conditioner 210 may be encapsulated by the fluid 226.

In some embodiments, the plurality of electrical components may be located on a substrate 230. The substrate 230 may be connected to the first surface 202 of the sensing device 200. For example, the substrate 230 and electrical components may be a system-on-chip and may be affixed to an interior side of the first surface 202. In another example, the substrate 230 may be the first surface 202 of the sensing device 200. In yet another example, the substrate 230 may be encapsulated by the fluid 226 inside the chamber 206. In other words, the substrate 230 and connected electrical components may be substantially surrounded by the fluid 226.

Sensing device 200 may be located in and/or laterally adjacent to a support member 232. In some embodiments, the support member 232 may be made of or include a compressible and/or elastically deformable material, such as open-cell foam, closed-cell foam, quilting, organic materials, spring (e.g., metal or plastic coil or leaf springs), other deformable materials, or combinations thereof. For example, the sensing device 200 may be located in a well 234 in a support member 232. In some embodiments, the well 234 may laterally surround the sensing device about 360°. In other embodiments, the well 234 may laterally surround the sensing device about less than 360°.

The well 234 may have a depth 236 from an outer surface 238 of the support member 232 that is no more than the height 222 of the sensing device 200. For example, at least a portion of the sensing device 200 may protrude from the well 234 beyond the outer surface 238 of the support member 232. The support member 232 may have a limit to its compressibility and create a mechanical ground for forces applied to the sensing device 200. The sensing device 200 may stand proud of the support member 232 to receive applied forces for a larger range of deformation of the sensing device 200 and/or support member 232.

In a relaxed state without force applied to the sensing device and/or support member, the sensing device 200 may have a height 222 that is greater than the depth 236 of the well 234. In some embodiments, a percentage of the height 222 of the sensing device 200 that is above the outer surface 238 of the support member 232 may be in a range having upper and lower values including any of 5%, 10%, 20%, 30%, 40%, 50%, 60%, 70%, 80%, 90%, or any value therebetween. For example, 5% to 90% of the height 222 of the sensing device 200 may be above the outer surface 238 of the support member 232. In other examples, 10% to 80% of the height 222 of the sensing device 200 may be above the outer surface 238 of the support member 232. In yet other examples, 20% to 70% of the height 222 of the sensing device 200 may be above the outer surface 238 of the support member 232. In further examples, 5% to 25% of the height 222 of the sensing device 200 may be above the outer surface 238 of the support member 232.

Referring now to FIG. 3, in some embodiments, in a relaxed state without force applied to a sensing device 300 and/or a support member 332, a portion of an area 340 of the second surface 304 of the sensing device 300 (when viewed from above) protruding beyond the outer surface 338 of the support member 332 may be in a range having upper and lower values including any of 10%, 20%, 30%, 40%, 50%, 60%, 70%, 80%, 90%, 100%, or any value therebetween. For example, 10% to 100% of the area 340 of the second surface 304 may protrude above the outer surface 338 of the support member 332. In other examples, 20% to 90% of the area 340 of the second surface 304 may protrude above the outer surface 338 of the support member 332. In yet other examples, 30% to 80% of the area 340 of the second surface 304 may protrude above the outer surface 338 of the support member 332.

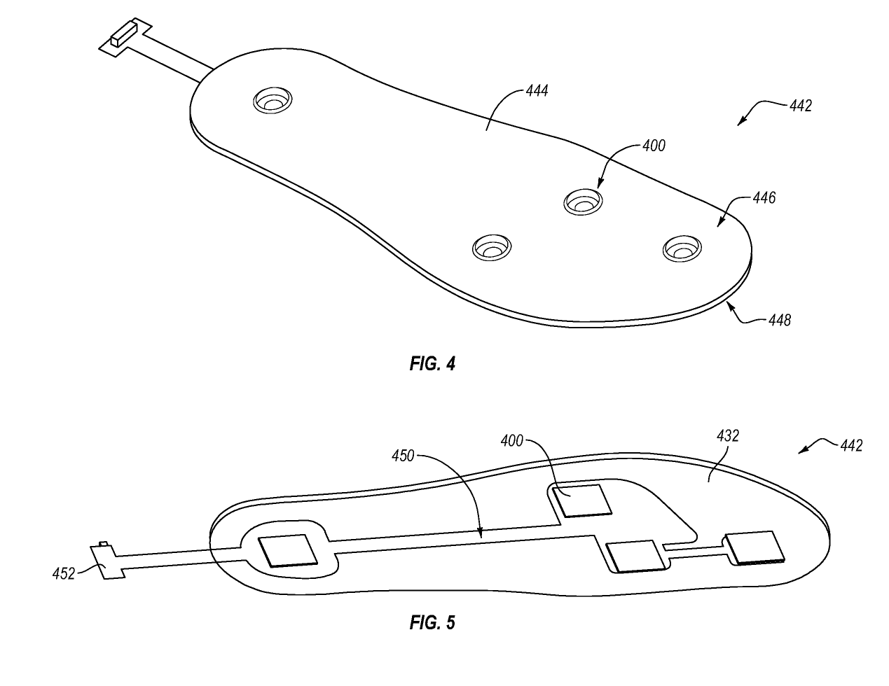

As shown in FIG. 4, a support member 432 may be a contact surface 444 of a therapeutic device 442. In some embodiments, the contact surface 444 may be a surface against which a portion of the patient's body may exert a force during usage of the therapeutic device 442. For example, one or more sensing devices 400 may be located on or in a contact surface 444 of a PWB device. In at least one example, the sensing device 400 may be located on or in a substantially planar outer surface, such as of a footbed in a PWB walking cast, as shown in FIG. 4. In other embodiments, the sensing device 400 may be located to receive a compressive force during attachment of the therapeutic device 442 to measure attachment forces. For example, the sensing device 400 may be located on or in a contact surface 444 of a prosthetic device.

In some embodiments, a therapeutic device 442 may include a support member 432 having a plurality of sensing devices 400 according to the present disclosure incorporated into an outer surface 438 thereof. For example, the support member 432 may be a footbed made of or including a compressible material. The support member 432 may have an upper 446 surface and a lower surface 448. At least one of the sensing devices 400 may be recessed into a well 434 in the upper surface 446 of the support member 432.

As shown in FIG. 5, in some embodiments, the support member 432 may include a flexible circuit 450 that allows electrical communication between the sensing devices 400 in the support member 432. The flexible circuit 450 may further include an electrical connector 452 that may provide electrical and/or data communication (i.e. conductive wiring and/or optical data transfer) to other sensing devices, communication modules, power supplies, data storage devices, other computing devices or combinations thereof. In other embodiments, one or more of the sensing devices 400 may include a wireless communication module providing data communication to other sensing devices, communication modules, power supplies, data storage devices, other computing devices or combinations thereof.

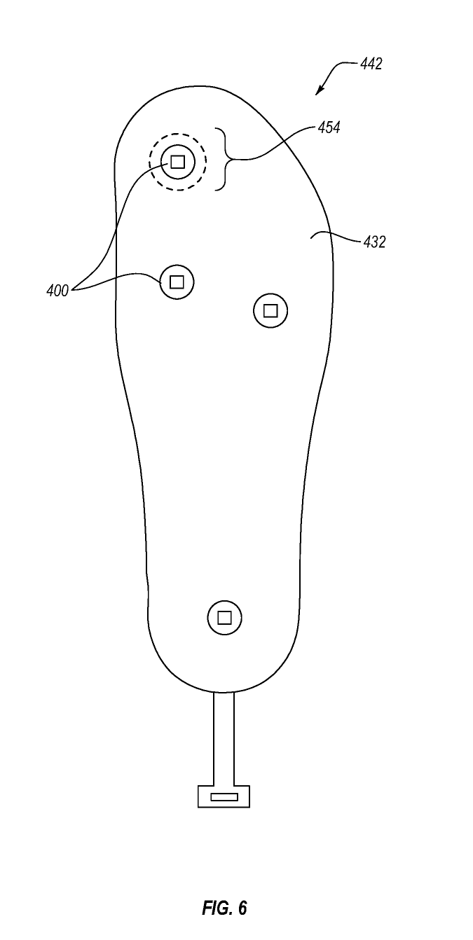

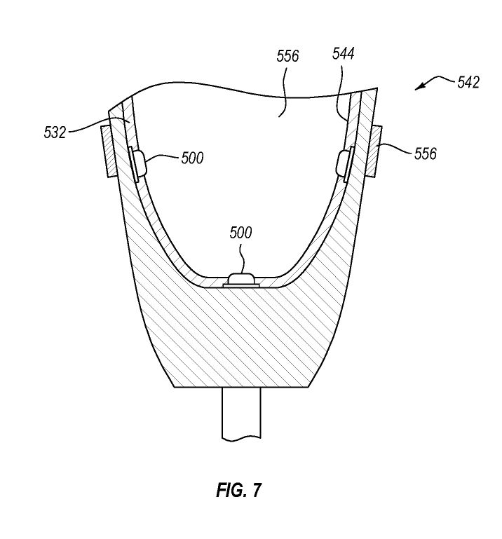

Referring now to FIG. 6, in some embodiments, the therapeutic device 442 may include a quantity of electrically connected sensing devices 400 in a range having upper and lower values including any of 2, 3, 4, 6, 8, 10, 12, 16, 20, or any values therebetween. Each of the sensing devices 400 may receive a force applied within a sensing area 454 including the area of the sensing device 400 and an area around the sensing device 400. The area of the sensing area 454 may be at least partially dependent upon the bulk modulus (i.e. compressibility) and/or thickness of the support member 432 adjacent the sensing device 400. For example, a less compressible support member 432 may mechanically ground the sensing device 400 at a lower applied force than a more compressible support member 432. In another example, a thinner support member 432 may mechanically ground the sensing device 400 at a lower applied force than a thicker support member 432.

Mechanically grounding the sensing device 400 may occur when the force applied to the support member 432 equals or exceeds the force applied to the sensing device. For example, the sensing device 400 may protrude from the support member 432 and, hence, contact a patient's foot without the foot contacting the support member 432. A movement of the foot toward the sensing device 400 may apply a force to the sensing device 400 and/or begin to deform the sensing device 400. The foot may continue to move toward the sensing device 400 and contact the support member 432, the support member 432 may apply a reactive force to the patient's foot and distribute the force, lowering the pressure on the sensing device 400 and limiting the nominal force applied thereto.

For example, a more compressible support member 432 may allow a greater proportion of the force applied by the foot to be applied to the sensing device 400. In other words, a less compressible or incompressible support member 432 may receive a greater proportion of the force applied by the foot and mechanically ground the foot, inhibiting further force being applied to the sensing device 400. In another example, a thicker support member 432 and/or a support member 432 having a greater compressible displacement may allow a greater proportion of the force applied by the foot to be applied to the sensing device 400. A thicker support member 432 and/or a support member 432 having a greater compressible displacement may continue to compress a greater distance (i.e., apply a lesser reactive force to the patient's foot) for a greater displacement than a thinner support member 432 and/or a support member 432 having a smaller compressible displacement. In other words, a thicker support member 432 and/or a support member 432 having a greater compressible displacement may displace farther than a thinner support member 432 and/or a support member 432 having a smaller compressible displacement before the portion of the support member 432 adjacent to the sensing device 400 “bottoms out” and ceases to compress further.

In some embodiments, the plurality of sensing devices 400 may be arranged in the support member 432 to correlate to bones, joints, or other areas of the patient's body that may, themselves be less compressible than other areas of the patient's body, thereby providing more direct transmission of force from the patient's body to the sensing device 400. For example, the sensing devices 400 in the therapeutic device 442 may be arranged to align with the major bones of the foot. The major bones may apply a greater proportion of the force from the foot to the surface upon which the patient stands to balance the patient. For example, the sensing devices 400 may be aligned with the calcaneus, the distal phalanx of the first phalanx, the joint of the first metatarsal and the first phalanx, the joint of the fifth metatarsal and the fifth phalanx, other bones and/or joints, or combinations thereof.

While an embodiment is shown and described in relation to a patient's foot, a therapeutic device including a support member and one or more sensing devices may be configured for measuring contact forces between other types of therapeutic devices, such as a prosthetic device, and a patient's residual limb. Referring now to FIG. 7, a contact surface 544 of the prosthetic device 542 may be or include a support member 532 and one or more sensing devices 500. For example, the contact surface 544 may be an inner surface of the prosthetic device 542, which may contact the residual limb 556. The contact surface 544 may be compressed against the residual limb 556 using hook-and-loop fastener straps 556, buckles, snaps, ratchet connections, adhesives, medical tape, bandages, other fasteners, or combinations thereof.

The prosthetic device 542 may provide improved comfort and/or control for the patient when compressed with a prescribed amount of force and/or the compressive force is distributed in a prescribed manner. The sensing devices 500 may provide the patient or medical professional localized information regarding the forces applied between the contact surface 544 and the residual limb 556. The forces between the contact surface 544 and the residual limb 556 may change over the duration of a day or other session of using the prosthetic device 542. The sensing devices 500 may provide a patient or medical professional localized information regarding the forces applied between the contact surface 544 and the residual limb 556 over the course of the day or other session of using the prosthetic device 542 and allow improved comfort and/or control of the prosthetic device 542.

At least one of the embodiments of a sensing device described herein may be used in other applications than presently described. In other applications, the sensing device may be used in hospital beds to measure pressure on the body to prevent bed sores or other ailments. In other examples, some embodiments of sensing devices may be utilized in athletic training or analysis to measure force distributions and/or balance of an athlete during particular activities, such as cycling, weight lifting, or running. Other embodiments may be utilized in monitoring forces applied to a body during physical activities in environments in which communication is limited, such as firefighting, policing, or industrial applications. Yet other embodiments of sensing devices may be utilized in monitoring forces and/or pressures in non-human applications, such as veterinary applications.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

1. A sensing device comprising:
a chamber defined by one or more membranes;
a pressure sensor located in the chamber;
a signal conditioner located in the chamber and in electrical communication with the pressure sensor; and
an incompressible fluid located in the chamber, the incompressible fluid contacting at least a first side of the pressure sensor and the one or more membranes.

2. The sensing device of claim 1, the incompressible fluid contacting at least one side of the signal conditioner.

3. The sensing device of claim 2, the incompressible fluid encapsulating the signal conditioner.

4. The sensing device of claim 1, the one or more membranes having an aperture therethrough, the aperture being located in communication with a second side of the pressure sensor, the second side of the pressure sensor being opposite the first side of the pressure sensor.

5. The sensing device of claim 1, the one or more membranes including a rigid first surface and a flexible second surface.

6. The sensing device of claim 5, further comprising a deformable sidewall connecting the rigid first surface to the flexible second surface.

7. The sensing device of claim 5, the rigid first surface and flexible second surface being directly connected to one another.

8. The sensing device of claim 1, further comprising a system on chip that includes the pressure sensor and signal conditioner.

9. A sensing device comprising:
a rigid first surface;
a flexible second surface that is at least partially convex relative to the rigid first surface, the rigid first surface and flexible second surface defining a chamber at least partially bounded by the rigid first surface and flexible second surface;
a pressure sensor located in the chamber; and
an incompressible fluid located in the chamber, contacting at least one side of the pressure sensor, the rigid first surface, and the flexible second surface. .

10. The sensing device of claim 9, the pressure sensor being a piezoresistive sensor.

11. The sensing device of claim 9, the flexible second surface being entirely convex relative to the rigid first surface.

12. The sensing device of claim 9, the sensing device having a height that is in a range of 10% to 100% of a width of the sensing device.

13. A therapeutic device comprising:
a flexible support member having an outer surface and an inner surface;
a plurality of wells in the outer surface of the support member extending from the outer surface toward the inner surface; and
a plurality of sensing devices located in the plurality of wells and in electrical communication with one another, at least one of the plurality of sensing devices including:
a rigid first surface,
a flexible second surface that is at least partially convex relative to the rigid first surface, the rigid first surface and flexible second surface defining a chamber at least partially bounded by the rigid first surface and flexible second surface,
a pressure sensor located in the chamber, and
an incompressible fluid located in the chamber, contacting at least one side of the pressure sensor.

14. The therapeutic device of claim 13, at least one of the plurality of sensing devices protruding beyond the outer surface of the flexible support member.

15. The therapeutic device of claim 14, the at least one of the plurality of sensing devices having a height, wherein 5% to 90% of the height of the at least one of the plurality of sensing devices protrudes from outer surface of the flexible support member.

16. The therapeutic device of claim 13, the flexible second surface defining an area of the at least one of plurality of sensing devices, and a range of 10% to 100% of the area of the at least one of the sensing device being beyond the outer surface of the flexible support member.

17. The therapeutic device of claim 13, further comprising an electrical connector in electrical communication with the plurality of sensing devices.

18. The therapeutic device of claim 13, the at least one of the plurality of sensing devices further including a signal conditioner located in the chamber and in electrical communication with the pressure sensor.

19. The therapeutic device of claim 13, the flexible second surface being convex relative to the rigid first surface.

20. The therapeutic device of claim 19, the rigid first surface being substantially planar and the rigid first surface and flexible second surface being connected to one another about a periphery of the rigid first surface and flexible second surface.
